(19) United States
(12) Patent Application Publication
(10) Pub. No.: US 2022/0153704 A1
(43) Pub. Date: May 19, 2022

(54) CRYSTAL FORM E OF BULLEYACONITINE A, PREPARATION METHOD THEREFOR AND APPLICATION THEREOF

(71) Applicant: YUNNAN HAOPY PHARMACEUTICALS LTD, Kunming (CN)

(72) Inventors: Qiongfen WU, Kunming (CN); Biao LI, Kunming (CN)

(21) Appl. No.: 17/438,753

(22) PCT Filed: Feb. 21, 2020
(86) PCT No.: PCT/CN2020/076156

§ 371 Date: Sep. 13, 2021

(30) Foreign Application Priority Data
Mar. 15, 2019 (CN) 201910197746.9

(51) Int. Cl. C07D 221/22

(52) U.S. Cl. CPC C07D 221/22; C07B 2200/13

(57) ABSTRACT

Provided is a crystal form E of bulleyaconitine A and a preparation method for the crystal form E of bulleyaconitine A. An X-ray powder diffraction spectrum of the crystal form measured by Cu-Kα-ray is as shown in FIG. 1. The crystal form E of bulleyaconitine A is prepared by adding a mixed solution of alcohol and water to bulleyaconitine A, stirring to obtain a suspended solid, and centrifugally collecting the solid. The alcohol is methanol, ethanol or n-butanol. The preparation process is simple, and the obtained crystal form has high purity and is characterized by XRD, DSC, TGA, and ¹HNMR to be determined as the crystal form E. The obtained bulleyaconitine A crystal is an anhydrous crystal form, and stability test results show that the crystal has good light, humidity and heat stability.

This application claims the priority of Chinese Patent Application No. 201910197746.9, filed with the China National Intellectual Property Administration on Mar. 15, 2019, and titled with “CRYSTAL FORM E OF BULLEYACONITINE A, PREPARATION METHOD THEREFOR AND APPLICATION THEREOF”, and the disclosures of which are hereby incorporated by reference.

### FIELD

The present disclosure relates to the field of medicinal chemistry, specifically to a crystalline form E of bulleyaconitine A, a preparation method therefor and an application thereof.

### BACKGROUND

Bulleyaconitine A has a chemical name of (1α,6α,14α,16β)tetrahydro-8,13,14-triol-20-ethyl-1,6,16-trimethoxy-4-methoxymethyl-8-acetoxy-14-(4′-p-methoxybenzyl)-aconitane. It is a diterpene diester alkaloid extracted and isolated from the root tuber of Aconitum georgei Comber, a plant of the genus Aconitum in the Ranunculaceae family, named Crassicauline A, and later, it was renamed Bulleaconitine A (T2). It is a known natural compound in plant species, and its structural formula is as follows:

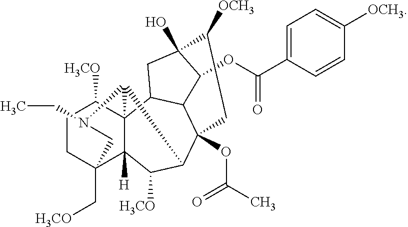

At present, bulleyaconitine A preparations are widely used clinically to treat rheumatoid arthritis (RA), osteoarthritis, myofibrositis, pain in neck and shoulder, pain in lower extremities and waist, cancerous pain and chronic pain caused by various reasons.

Polymorphism in pharmaceuticals is a common phenomenon in drug research and development, and is an important factor which influences drug quality. The same drugs with different crystalline forms vary in appearance, solubility, melting point, dissolution, and bioavailability, and may even have significant differences. Therefore, the crystalline form of the drug will affect the stability, bioavailability and therapeutic effect. Moreover, the crystalline form of a drug will also affect the quality and absorption behavior in human body of a pharmaceutical preparation of the drug, and finally affects the benefit ratio between the therapeutic effect and side effect of the preparation in human body. With the in-depth research of bulleyaconitine A, the research on the crystalline form and physicochemical properties of bulleyaconitine A is of great significance to the evaluation of the drug efficacy, quality, and safety of bulleyaconitine A. The Chinese patent with application number 201710423005.9 discloses that bulleyaconitine A is dissolved with a C1-4 organic solvent, then the obtained bulleyaconitine A solution is added dropwise to water, stirring while adding, and after the addition, suction filtration is performed and the filter cake is dried to obtain the amorphous bulleyaconitine A. So far, there is no relevant report on crystalline bulleyaconitine A.

### SUMMARY

In view of this, the purpose of the present disclosure is to provide a new crystalline form of bulleyaconitine A and a preparation method thereof.

An object of the present disclosure is to research, discover and provide the crystalline form E of bulleyaconitine A by crystallographic methods.

In the present disclosure, X-ray powder diffraction (XRPD), which is internationally acknowledged, is adopted to study and characterize the crystalline form of bulleyaconitine A. Measurement conditions and methods: Cu/K-alpha1 (target), 45 KV-40 mA (working voltage and current), 2θ=3-40 (scanning range), scanning time per step (s) is 17.8-46.7, scanning step size (2θ) is 0.0167-0.0263, λ=1.54 Å.

The substantially pure crystalline form E of bulleyaconitine A provided by the present disclosure has an X-ray powder diffraction spectrum as shown in FIG. 1, and its X-ray powder diffraction spectrum shows obvious characteristic absorption peaks at 2θ values of 7.8±0.2, 9.4±0.2, 11.5±0.2, 12.4±0.2, 13.2±0.2, 13.8±0.2, 14.8±0.2, 16.6±0.2, 18.8±0.2, 19.3±0.2, 22.1±0.2, and 33.6±0.2.

The present disclosure also adopts thermogravimetric analysis to study and characterize the crystalline form E of bulleyaconitine A. The detection conditions are: starting from room temperature, and the heating gradient is heating up to 400° C. at a rate of 10° C./min, with nitrogen as the protective gas.

The substantially pure crystalline form E of bulleyaconitine A provided by the present disclosure has a thermogravimetric analysis graph as shown in FIG. 2, and it has the following characteristics: when the temperature rises to 150° C., the sample has a weight loss of 0.3%.

The present disclosure also adopts differential scanning calorimetry to study and characterize the crystalline form E of bulleyaconitine A. The detection method is: starting from 25° C., and the temperature rise gradient is increasing temperature to 280° C. at a rate of 10° C./min, with nitrogen as the protective gas.

The substantially pure crystalline form E of bulleyaconitine A provided by the present disclosure has a differential scanning calorimetry graph as shown in FIG. 2, and it has the following characteristics: the endothermic peak is 160-164° C.

It is worth noting that among the X-ray powder diffraction spectra of the above-mentioned crystalline form, the characteristic peaks of the X-ray powder diffraction spectrum may have slight differences between one machine and another machine and between one sample and another sample. The value may differ by about 1 unit, or by about 0.8 unit, or by about 0.5 unit, or by about 0.3 unit, or by about 0.1 unit, so the value given should not be regarded as absolute. Similarly, the values given in the differential scanning calorimetry graphs of the above-mentioned crystalline forms should not be regarded as absolute either.

The crystalline form can also be characterized by other analytical techniques known in the art, such as hydrogen nuclear magnetic resonance spectrum (¹HNMR), polarized light microscopy (PLM), and dynamic vapor sorption (DVS).

The substantially pure crystalline form E of bulleyaconitine A provided by the present disclosure has a hydrogen nuclear magnetic resonance spectrum as shown in FIG. 3, polarized light microscopy analysis graph as shown in FIG. 4, and dynamic vapor sorption graph as shown in FIG. 5.

The present disclosure also provides a preparation method of the crystalline form E of bulleyaconitine A with high purity and no residual solvent.

The preparation method of the crystalline form E of bulleyaconitine A provided by the present disclosure comprises adding a mixed solution of alcohol and water to bulleyaconitine A, stirring to obtain a suspended solid, and centrifugally collecting the solid; wherein the alcohol is methanol, ethanol or n-butanol.

Preferably, the volume ratio of alcohol to water in the mixed solution of alcohol and water in the preparation method of the crystalline form E of bulleyaconitine A is 10:1-1:10.

Preferably, in mg/ml, the ratio of the bulleyaconitine A to the mixed solution of alcohol and water of the present disclosure is 3:1-1000:1.

Preferably, the stirring time in the preparation method of the crystalline form E of bulleyaconitine A of the present disclosure is at least 0.5 hours.

Preferably, the stirring temperature in the preparation method of the crystalline form E of bulleyaconitine A of the present disclosure is 0° C.-50° C.

The crystalline form E of bulleyaconitine A obtained by the preparation method of the present disclosure has a crystalline form content of more than 99%, high purity, consistent X-ray powder diffraction spectrum characteristics and DSC characteristics, stable properties, and good stability to light, humidity and heat.

The present disclosure also provides use of the crystalline form E of bulleyaconitine A in the manufacture of a medicament for the prevention and/or treatment of rheumatoid arthritis (RA), osteoarthritis, myofibrositis, pain in neck and shoulder, pain in lower extremities and waist, or cancerous pain.

It can be known from the above technical solutions that the present disclosure discloses a crystalline form E of bulleyaconitine A and a preparation method thereof. An X-ray powder diffraction spectrum of the crystalline form of the present disclosure measured by Cu-Kα-ray is as shown in FIG. 1. The crystalline form E of bulleyaconitine A is prepared by adding a mixed solution of alcohol and water to bulleyaconitine A, stirring to obtain a suspended solid, and centrifugally collecting the solid; wherein the alcohol is methanol, ethanol or n-butanol. The preparation process is simple, and the obtained crystalline form has high purity and is characterized by XRD, DSC, TGA, and ¹HNMR to be determined as the crystalline form E. The obtained crystalline form E of bulleyaconitine A is an anhydrous crystalline form, and stability test results show that the crystal has good light, humidity and heat stability.

### BRIEF DESCRIPTION OF DRAWINGS

In order to more clearly illustrate the technical solutions in the examples of the present disclosure or in the prior art, the drawings used in the examples or the prior art will be briefly introduced below.

FIG. 1 XRPD graph of the crystalline form E of bulleyaconitine A;

FIG. 2 TGA/DSC graph of the crystalline form E of bulleyaconitine A;

FIG. 3 ¹H NMR spectrum of the crystalline form E of bulleyaconitine A;

FIG. 4 PLM graph of the crystalline form E of bulleyaconitine A;

FIG. 5 DVS graph of the crystalline form E of bulleyaconitine A;

FIG. 6 XRPD comparison graph before and after DVS test of the crystalline form E of bulleyaconitine A;

FIG. 7 XRPD comparison graph before and after stability evaluation of the crystalline form E of bulleyaconitine A.

### DETAILED DESCRIPTION

Hereinafter, the technical solutions in embodiments of the present disclosure will be described clearly and completely in conjunction with examples of the present disclosure. It is apparent that the described examples are merely part of the present disclosure rather than all. Based on the examples in the present disclosure, all other examples obtained by those of ordinary skill in the art without creative work are within the scope of the present disclosure.

The present disclosure will be illustrated in detail in combination with specific examples below in order to further understand the present disclosure. In the following examples, unless otherwise specified, the test method is usually implemented in accordance with conventional conditions or conditions recommended by the manufacturer.

### Test Parameters

The XRPD patterns were collected on PANalytacal Empyrean and X' Pert3 X-ray powder diffraction analyzers. The scanning parameters are shown in Table 1.

TABLE 1
XRPD test parameters

| Parameters | Reflection mode | | | Transmission mode |
|---|---|---|---|---|
| Instrument model | Empyrean | X′ Pert3 | X′ Pert3 | X′ Pert3 |
| X-ray | Cu, kα, Kα1 (Å): 1.540598, Kα2 (Å): 1.544426; | | | |
| | Kα2/Kα1 intensity ratio: 0.50 | | | |
| X-ray tube setting | 45 kV, 40 mA | | | |
| Divergence slit | Automatic | Fixed ⅛° | Fixed ⅙° | Fixed ½° |
| Scanning mode | Continuous | | | |
| Scanning range (°2Theta) | 3~40 | | | |
| Scanning time per step (s) | 17.8 | 46.7 | | 33.02 |
| Scanning step size (°2Theta) | 0.0167 | 0.0263 | | 0.0167 |
| Test time | 5 min 30 s | 5 min 4 s | | 10 min 11 s |

### Thermogravimetric Analysis (TGA) and Differential Scanning Calorimetry (DSC)

TGA and DSC graphs were collected on TA Q5000 TGA/TA Discovery TGA5500 thermogravimetric analyzer and TA Q2000 DSC/TA Discovery DSC2500 differential scanning calorimeter, respectively. Table 2 lists the test parameters.

TABLE 2
TGA and DSC test parameters

| Parameters | TGA | DSC |
|---|---|---|
| Method | Linear heating | Linear heating |
| Sample pan | Aluminum pan, open | Aluminum pan, gland |
| Temperature range | Room temperature- | 25° C.- |
| | End temperature set | End temperature set |
| Scanning rate (° C./min) | 10 | 10 |
| Protective gas | Nitrogen | Nitrogen |

### Dynamic Vapor Sorption (DVS)

The dynamic vapor sorption (DVS) graph was collected on DVS Intrinsic of SMS (Surface Measurement Systems). The relative humidity at 25° C. was corrected by the deliquescent point of LiCl, Mg(NO₃)₂ and KCl. DVS test parameters are listed in Table 3.

TABLE 3
DVS test parameters

| Parameter | Set value |
|---|---|
| Temperature | 25° C. |
| Sample size | 10-20 mg |
| Protective gas and flow | N₂, 200 ml/min |
| rate | |
| dm/dt | 0.002%/min |
| Minimum dm/dt balance | 10 min |
| time | |
| Maximum balance time | 180 min |
| RH range | 0% RH-90% RH-0% RH |
| RH gradient | 10%(0% RH-90% RH, 90% RH-0% RH) |
| | 5%(90% RH-95% RH, 95% RH-90% RH) |

### Liquid NMR

The liquid NMR spectra were collected on Bruker 400M NMR spectrometer, with DMSO-d6 as the solvent.

### Example 1. Preparation and Identification of Crystalline Form E of Bulleyaconitine A

15 mg of bulleyaconitine A was weighed out and placed in a 3 ml vial. Then 0.5 ml of n-butanol-water (1:1) was added, and stirred for 2 hours at 5° C. Centrifugation was performed to separate and obtain a solid. The solid was subjected to XRPD, TGA/DSC and ¹HNMR tests.

The XRPD results show that there are obvious characteristic absorption peaks at the diffraction angle (2θ angle) of 7.6±0.2, 9.4±0.2, 11.3±0.2, 12.4±0.2, 13.4±0.2, 13.9±0.2, 14.8±0.2, 16.8±0.2, 18.8±0.2, 19.4±0.2, 22.2±0.2, and 33.1±0.2. The TGA/DSC results show that when the temperature rises to 150° C., the weight loss is 0.3%, and the DSC graph shows a sharp endothermic peak at 160.9° C. (initial temperature), which may be caused by melting. Combined with the TGA weight loss, it is speculated that the thermal signal appearing above 200° C. on the DSC is caused by the decomposition of the sample. ¹HNMR results show that there is no obvious solvent residue in the sample. The PLM results show a composition of irregular small particles.

It was identified as crystalline form E, anhydrous crystal.

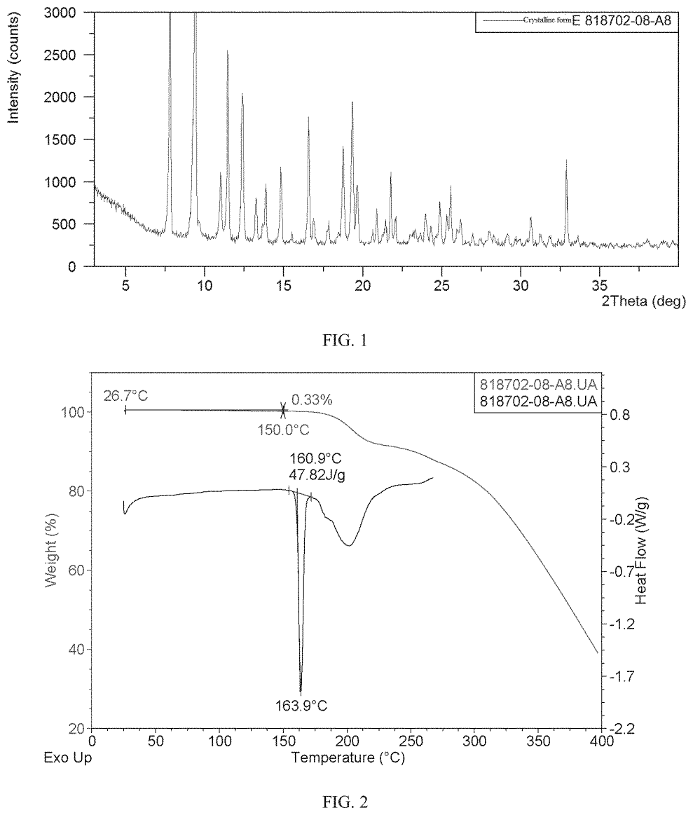

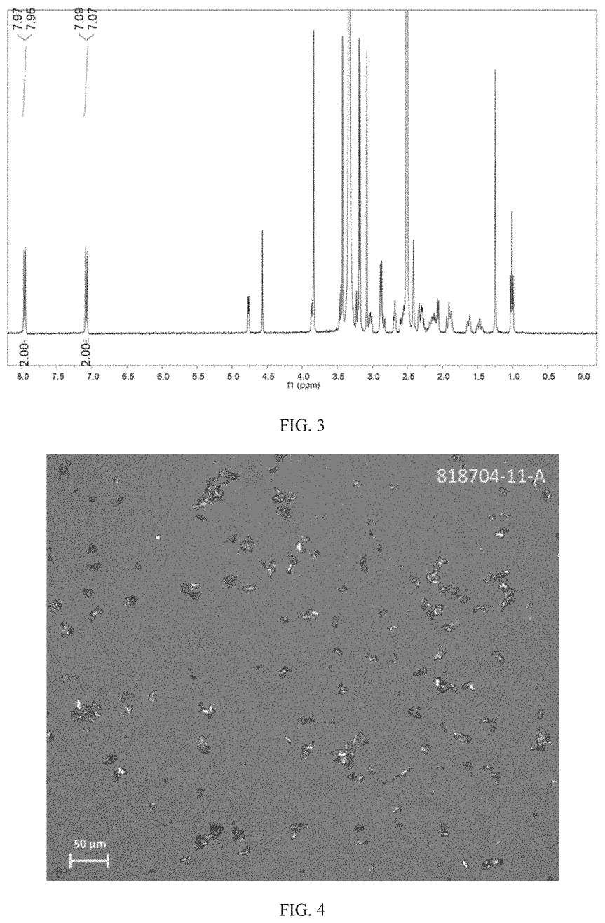

For the sample, the XRPD graph is shown in FIG. 1, the TGA/DSC characterization result graph is shown in FIG. 2, the ¹HNMR graph is shown in FIG. 3, and the PLM result graph is shown in FIG. 4.

### Example 2. Preparation of Crystalline Form E of Bulleyaconitine A

15 mg of bulleyaconitine A was weighed out and placed in a 3 ml vial. Then 0.5 ml of n-butanol-water (1:10) was added, and stirred for 0.5 hours at 25° C. Centrifugation was performed to separate and obtain a solid. The solid was subjected to XRPD test, and the results are consistent with FIG. 1 that it was determined to be the crystalline form E of bulleyaconitine A; the DSC graph shows an endothermic peak of 163.9° C.

### Example 3. Preparation of Crystalline Form E of Bulleyaconitine A

15 mg of bulleyaconitine A was weighed out and placed in a 3 ml vial. Then 0.3 ml of n-butanol-water (1:1) was added, and stirred for 3 hours at 50° C. Centrifugation was performed to separate and obtain a solid. The solid was subjected to XRPD test, and the results are consistent with FIG. 1 that it was determined to be the crystalline form E of bulleyaconitine A; the DSC graph shows an endothermic peak of 160° C.

### Example 4. Preparation of Crystalline Form E of Bulleyaconitine A

15 mg of bulleyaconitine A was weighed out and placed in a 3 ml vial. Then 0.3 ml of n-butanol-water (1:10) was added, and stirred for 1 hours at 25° C. Centrifugation was performed to separate and obtain a solid. The solid was subjected to XRPD test, and the results are consistent with FIG. 1 that it was determined to be the crystalline form E of bulleyaconitine A; the DSC graph shows an endothermic peak of 164° C.

### Example 5. Preparation of Crystalline Form E of Bulleyaconitine A

1500 mg of bulleyaconitine A was weighed out and placed in a beaker. Then 15 ml of n-butanol-water (5:1) was added, and stirred for 5 hours at 15° C. Centrifugation was performed to separate and obtain a solid. The solid was subjected to XRPD test, and the results are consistent with FIG. 1 that it was determined to be the crystalline form E of bulleyaconitine A; the DSC graph shows an endothermic peak of 162.9° C.

### Example 6. Preparation of Crystalline Form E of Bulleyaconitine A

100 mg of bulleyaconitine A was weighed out and placed in a beaker. Then 1 ml of n-butanol-water (1:8) was added, and stirred for 10 hours at 5° C. Centrifugation was performed to separate and obtain a solid. The solid was subjected to XRPD test, and the results are consistent with FIG. 1 that it was determined to be the crystalline form E of bulleyaconitine A; the DSC graph shows an endothermic peak of 161.5° C.

### Example 7. Preparation of Crystalline Form E of Bulleyaconitine A

1000 mg of bulleyaconitine A was weighed out and placed in a beaker. Then 1 ml of n-butanol-water (10:1) was added, and stirred for 24 hours at 0° C. Centrifugation was performed to separate and obtain a solid. The solid was subjected to XRPD test, and the results are consistent with FIG. 1 that it was determined to be the crystalline form E of bulleyaconitine A; the DSC graph shows an endothermic peak of 163.1° C.

### Example 8. Preparation of Crystalline Form E of Bulleyaconitine A

15 mg of bulleyaconitine A was weighed out and placed in a 3 ml vial. Then 0.5 ml of methanol-water (1:10) was added, and stirred for 0.5 hours at 25° C. Centrifugation was performed to separate and obtain a solid. The solid was subjected to XRPD test, and the results are consistent with FIG. 1 that it was determined to be the crystalline form E of bulleyaconitine A; the DSC graph shows an endothermic peak of 161.7° C.

### Example 9. Preparation of Crystalline Form E of Bulleyaconitine A

15 mg of bulleyaconitine A was weighed out and placed in a 3 ml vial. Then 0.3 ml of methanol-water (1:1) was added, and stirred for 3 hours at 50° C. Centrifugation was performed to separate and obtain a solid. The solid was subjected to XRPD test, and the results are consistent with FIG. 1 that it was determined to be the crystalline form E of bulleyaconitine A; the DSC graph shows an endothermic peak of 162.6° C.

### Example 10. Preparation of Crystalline Form E of Bulleyaconitine A

15 mg of bulleyaconitine A was weighed out and placed in a 3 ml vial. Then 0.3 ml of methanol-water (1:10) was added, and stirred for 1 hours at 25° C. Centrifugation was performed to separate and obtain a solid. The solid was subjected to XRPD test, and the results are consistent with FIG. 1 that it was determined to be the crystalline form E of bulleyaconitine A; the DSC graph shows an endothermic peak of 161.8° C.

### Example 11. Preparation of Crystalline Form E of Bulleyaconitine A

1500 mg of bulleyaconitine A was weighed out and placed in a beaker. Then 15 ml of methanol-water (5:1) was added, and stirred for 5 hours at 15° C. Centrifugation was performed to separate and obtain a solid. The solid was subjected to XRPD test, and the results are consistent with FIG. 1 that it was determined to be the crystalline form E of bulleyaconitine A; the DSC graph shows an endothermic peak of 163.98° C.

### Example 12. Preparation of Crystalline Form E of Bulleyaconitine A

100 mg of bulleyaconitine A was weighed out and placed in a beaker. Then 1 ml of methanol-water (1:8) was added, and stirred for 10 hours at 5° C. Centrifugation was performed to separate and obtain a solid. The solid was subjected to XRPD test, and the results are consistent with FIG. 1 that it was determined to be the crystalline form E of bulleyaconitine A; the DSC graph shows an endothermic peak of 161.9° C.

### Example 13. Preparation of Crystalline Form E of Bulleyaconitine A

1000 mg of bulleyaconitine A was weighed out and placed in a beaker. Then 1 ml of methanol-water (10:1) was added, and stirred for 24 hours at 0° C. Centrifugation was performed to separate and obtain a solid. The solid was subjected to XRPD test, and the results are consistent with FIG. 1 that it was determined to be the crystalline form E of bulleyaconitine A; the DSC graph shows an endothermic peak of 160.2° C.

### Example 14. Preparation of Crystalline Form E of Bulleyaconitine A

15 mg of bulleyaconitine A was weighed out and placed in a 3 ml vial. Then 0.5 ml of ethanol-water (1:10) was added, and stirred for 0.5 hours at 25° C. Centrifugation was performed to separate and obtain a solid. The solid was subjected to XRPD test, and the results are consistent with FIG. 1 that it was determined to be the crystalline form E of bulleyaconitine A; the DSC graph shows an endothermic peak of 160.8° C.

### Example 15. Preparation of Crystalline Form E of Bulleyaconitine A

15 mg of bulleyaconitine A was weighed out and placed in a 3 ml vial. Then 0.3 ml of ethanol-water (1:1) was added, and stirred for 3 hours at 50° C. Centrifugation was performed to separate and obtain a solid. The solid was subjected to XRPD test, and the results are consistent with FIG. 1 that it was determined to be the crystalline form E of bulleyaconitine A; the DSC graph shows an endothermic peak of 163° C.

### Example 16. Preparation of Crystalline Form E of Bulleyaconitine A

15 mg of bulleyaconitine A was weighed out and placed in a 3 ml vial. Then 0.3 ml of ethanol-water (1:10) was added, and stirred for 1 hours at 25° C. Centrifugation was performed to separate and obtain a solid. The solid was subjected to XRPD test, and the results are consistent with FIG. 1 that it was determined to be the crystalline form E of bulleyaconitine A; the DSC graph shows an endothermic peak of 162° C.

### Example 17. Preparation of Crystalline Form E of Bulleyaconitine A

1500 mg of bulleyaconitine A was weighed out and placed in a beaker. Then 15 ml of ethanol-water (5:1) was added, and stirred for 5 hours at 15° C. Centrifugation was performed to separate and obtain a solid. The solid was subjected to XRPD test, and the results are consistent with FIG. 1 that it was determined to be the crystalline form E of bulleyaconitine A; the DSC graph shows an endothermic peak of 163.5° C.

### Example 18. Preparation of Crystalline Form E of Bulleyaconitine A

100 mg of bulleyaconitine A was weighed out and placed in a beaker. Then 1 ml of ethanol-water (1:8) was added, and stirred for 10 hours at 5° C. Centrifugation was performed to separate and obtain a solid. The solid was subjected to XRPD test, and the results are consistent with FIG. 1 that it was determined to be the crystalline form E of bulleyaconitine A; the DSC graph shows an endothermic peak of 162.4° C.

### Example 19. Preparation of Crystalline Form E of Bulleyaconitine A

1000 mg of bulleyaconitine A was weighed out and placed in a beaker. Then 1 ml of ethanol-water (10:1) was added, and stirred for 24 hours at 0° C. Centrifugation was performed to separate and obtain a solid. The solid was subjected to XRPD test, and the results are consistent with FIG. 1 that it was determined to be the crystalline form E of bulleyaconitine A; the DSC graph shows an endothermic peak of 161.6° C.

### Example 20. Stability Test of Crystalline Form E of Bulleyaconitine A

### 1) DVS Characterization of Crystalline Form E

In order to evaluate the hygroscopicity and stability of anhydrous crystalline form E under different humidity conditions, DVS and XRPD tests were performed on crystalline form E samples at a constant temperature of 25° C.

The crystalline form E continued to slowly adsorb water as the humidity increased. When the humidity reached 80% RH, 0.12% of water was adsorbed, indicating that the sample has no hygroscopicity. The XRPD characterization results show that the crystalline form of the crystalline form E sample before and after the DVS test did not change. It can be seen from the XRPD comparison results that the crystalline form of the sample did not change after the DVS test.

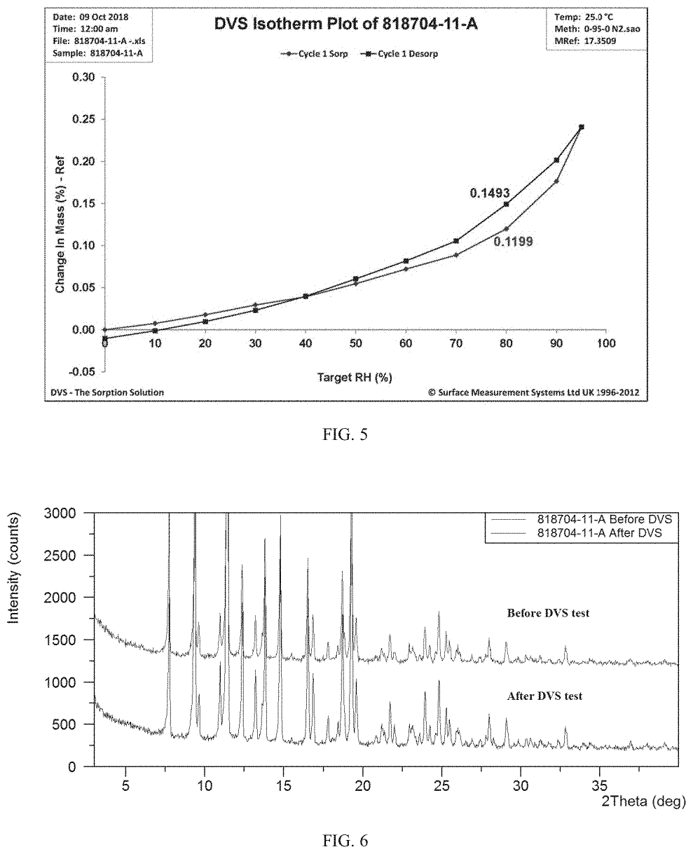

The DVS graph of the crystalline form E is shown in FIG. 5, and the XRPD comparison graph of the crystalline form E before and after the DVS test is shown in FIG. 6.

### 2) Evaluation of the Solid State Stability of Crystalline Form E

In order to evaluate the solid state stability of crystalline form E, an appropriate amount of samples was weigh out and placed in an open place at 25° C./60% RH and 40° C./75% RH for 1 week and 1 month, respectively, and placed in a sealed place at 80° C. for 24 hours. XRPD and HPLC characterization of the placed samples were performed to detect the changes of crystalline form and chemical purity.

The HPLC results are shown in Table 4 that the chemical purity of the sample has not changed under the selected test conditions; and the XRPD results show that the crystalline form of the sample has not changed under the selected test conditions.

TABLE 4
Summary of stability data of crystalline form E

| Crystalline | | | HPLC purity | | |
|---|---|---|---|---|---|
| form | | | Area | % of | Final crystalline |
| (Sample No.) | Conditions | | % | Control | form |
| Crystalline | 80° C., 24 hours | | 99.34 | 99.9 | Crystalline |
| form E | 25° C./ | 1 week | 99.48 | 100.0 | form E |
| | 60% RH | 1 month | 99.53 | 100.1 | |
| | 40° C./ | 1 week | 99.49 | 100.0 | |
| | 75% RH | 1 month | 99.47 | 100.0 | |

In conclusion, the crystalline form E has good physical and chemical stability.

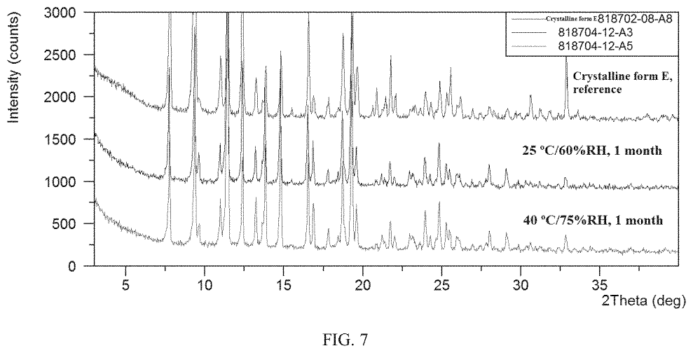

The XRPD comparison graph of the crystalline form E before and after the stability evaluation is shown in FIG. 7.

1. A crystalline form E of bulleyaconitine A, wherein its X-ray powder diffraction spectrum shows obvious characteristic absorption peaks at 2θ values of 7.8±0.2, 9.4±0.2, 11.5±0.2, 12.4±0.2, 13.2±0.2, 13.8±0.2, 14.8±0.2, 16.6±0.2, 18.8±0.2, 19.3±0.2, 22.1±0.2, and 33.6±0.2.

2. The crystalline form E of bulleyaconitine A according to claim 1, wherein its thermogravimetric analysis graph shows a weight loss of 0.3% when heated to 150° C.

3. The crystalline form E of bulleyaconitine A according to claim 1, wherein its differential scanning calorimetry analysis graph shows an endothermic peak at 160-164° C.

4. The crystalline form E of bulleyaconitine A according to claim 1, wherein its hydrogen nuclear magnetic resonance spectrum is shown in FIG. 3.

5. A preparation method of the crystalline form E of bulleyaconitine A according to claim 1, comprising adding a mixed solution of alcohol and water to bulleyaconitine A, stirring to obtain a suspended solid, and centrifugally collecting the solid; wherein the alcohol is methanol, ethanol or n-butanol.

6. The preparation method of the crystalline form E of bulleyaconitine A according to claim 5, wherein the volume ratio of alcohol to water in the mixed solution of alcohol and water is 10:1-1:10.

7. The preparation method of the crystalline form E of bulleyaconitine A according to claim 5, wherein, in mg/ml, the mass-volume ratio of the bulleyaconitine A to the mixed solution of alcohol and water is 3:1-1000:1.

8. The preparation method of the crystalline form E of bulleyaconitine A according to claim 5, wherein the stirring time is at least 0.5 hours.

9. The preparation method of the crystalline form E of bulleyaconitine A according to claim 5, wherein the stirring temperature is 0° C.-50° C.

10. A method for preventing and/or treating rheumatoid arthritis, osteoarthritis, myofibrositis, pain in neck and shoulder, pain in lower extremities and waist, or cancerous pain, comprising administering a therapeutically effective amount of the crystalline form E of bulleyaconitine A according to claim 1 to a subject in need thereof.
